(19) United States
(12) Patent Application Publication
(10) Pub. No.: US 2004/0185022 A1
(43) Pub. Date: Sep. 23, 2004

(54) Regimen for acne treatment

(76) Inventor: Steven Rubin, Branford, CT (US)

Correspondence Address: Peter DeLuca, Carter, DeLuca, Farrell & Schmidt, LLP, 445 Broad Hollow Road, Suite 225, Melville, NY, 11747, US

(21) Appl. No.: 10/781,261

(22) Filed: Feb. 18, 2004

Related U.S. Application Data
(60) Provisional application No. 60/448,688, filed on Feb. 19, 2003.

(51) Int. Cl. A61K 7/135; A61K 7/06; A61K 7/11

(52) U.S. Cl. 424/70.13; 514/54

(57) ABSTRACT

An acne treatment regimen includes: cleansing the skin of an individual afflicted with acne with an acne cleanser composition containing an anti-acne active; applying an acne spot treating composition to at least one lesion on the skin of the individual, the acne spot treating composition containing an anti-acne active ingredient; applying a hydrating day cream composition to the skin of the individual, the hydrating day cream composition containing an anti-acne active ingredient and a combination of farnesol and sodium hyaluronate; and applying an acne night cream composition to the skin of the individual, the acne night cream composition containing an anti-acne active ingredient and a combination of famesol and sodium hyaluronate.

### CROSS REFERENCE TO RELATED APPLICATION

This application claims priority to and the benefit of U.S. Provisional Application Serial No. 60/448,688 filed on Feb. 19, 2003, the contents of which are incorporated herein by reference.

### BACKGROUND

1. Technical Field

This disclosure relates to compositions useful for the treatment of acne. More specifically, this disclosure relates to a four step regimen for the treatment of acne.

2. Background of Related Art

Acne is a common inflammatory disease of human skin, and concentrates in skin areas where sebaceous glands are largest, most numerous, and most active. In its milder types, it is a more or less superficial disorder which is evidenced by slight, spotty irritations and ordinary skin hygiene is a satisfactory treatment. However, in the more inflammatory types of acne, bacterial invasion of or about the pilosebaceous follicles occurs and pustules, infected cysts and, in extreme cases, canalizing inflamed and infected sacs appear. These lesions may become extensive and leave permanent, disfiguring scars.

Acne is very common by puberty and at least 80% of teenagers are afflicted. The facial eruptions are known to cause such psychic trauma in many adolescents that they find it difficult to make personal adjustments and consequently, withdraw and self-pity occur. The sufferer may be constantly aware of the obvious facial blemishes. For these reasons a medicinal preparation and treatment are of definite benefit and may eliminate the need for psychotherapy.

To reduce the severity of acne, various forms of medication have previously been topically applied to the skin. Antibacterial soaps have been used as well as bactericidal agents such as sulfur and resorcinol. Other topical compositions have separately contained benzoyl peroxide, hexachlorophene, erythromycin or neomycin sulfate. None of these prior preparations has been completely effective.

### SUMMARY

A novel, four step regimen for treatment of acne is described herein. The regimen includes the use of an acne cleanser, an acne spot treatment, an acne hydrating day cream and an acne night cream. Each component of the regimen includes at least one anti-acne active. The spot treatment, acne hydrating day cream and the acne night cream each include a combination of farnesol and sodium hyaluronate.

In another aspect, a method of treating acne is described herein which includes applying to the skin of a person afflicted with acne an acne cleanser, an acne spot treatment, an acne hydrating day cream and an acne night cream, each of the components of the treatment being applied at least once a day.

In another aspect, the regimen is provided as a kit that includes an acne cleanser, an acne spot treatment, an acne hydrating day cream and an acne night cream all contained in a single package.

### BRIEF DESCRIPTION OF THE DRAWINGS

FIG. 1 shows the reduction in inflammatory lesions resulting from use of a regimen in accordance with the present disclosure.

FIG. 2 shows the percentage of patients that noticed a reduction of new lesions resulting from use of a regimen in accordance with the present disclosure.

FIG. 3 shows the percentage of patients that noticed a decrease in inflammation resulting from use of a regimen in accordance with the present disclosure.

FIG. 4 shows the percentage of patients that noticed a reduction in oiliness resulting from use of a regimen in accordance with the present disclosure.

### DETAILED DESCRIPTION OF PREFERRED EMBODIMENTS

The present four step regimen for acne treatment includes the use of an acne cleanser composition, an acne spot treatment composition, an acne hydrating day cream composition and an acne night cream composition. Each of the four compositions includes an anti-acne active ingredient.

As used herein, the term “anti-acne active ingredient” means any active ingredient which is effective in treating acne. Among the anti-acne actives presently known are peroxides (including benzoyl peroxide, stabilized hydrogen peroxide and peroxides of organic acids, such as a lauroyl peroxide), antibiotic or antibacterial (such as, for example, clindamycin, neomycin, sodium sulfacetamide, sulfur, tetracycline or erythromycin), salicylic acid and its derivatives (such as salts and esters). Combinations of materials can also be used as the anti-acne active ingredient. For example, a combination of sulfur and resorcinol or resorcinol monoacetate can be used. In particularly useful embodiments, the anti-acne active is salicylic acid. The anti-acne active ingredient will be present in each of the compositions used in the regimen in an amount form 0.01 to 10 percent by weight of each composition, preferably 0.1 to 5 percent by weight of each composition, most preferably 0.5 to 3 percent by weight of each composition.

The acne cleanser composition includes an anti-acne active and one or more cleansers.

Suitable cleansers include but are not limited to the synthetic surfactants. The surfactants can be selected from the group consisting of anionic, cationic, amphoteric and alkylglycosidic surface active agents. A preferred content of cleanser is between about 2-30 weight percent by weight of the acne cleanser composition.

Suitable anionic surfactants include, for example, alkyl and alkyl ether sulfates (such as sodium cocoalkyl triethylene glycol ether sulfate); water-soluble salts corresponding to the formula R₁—SO₃-M, where R₁ is a C₈-C₂₄ aliphatic group, and M is a cation; phosphates such as monoalkyl, dialkyl, and trialkylphosphate salts formed by the reaction of phosphorous pentoxide with monohydric branched or unbranched alcohols having from about 8 to about 24 carbon atoms (such as sodium mono or dilaurylphosphate); the reaction products of fatty acids esterified with isethionic acid and neutralized with an alkaline reagent; sulfonated fatty acids (such as alpha sulphonated coconut fatty acid and lauryl methyl ester); acyl isethionates (such as ammonium cocoyl isethionate, sodium cocoyl isethionate, sodium lauroyl isethionate); acyl glutamates (such as sodium lauroyl glutamate and sodium cocoyl glutamate); sulfosuccinate salts (such as disodium N-octadecylsulfosuccinamate and sodium dioctyl sulfosuccinate); carboxylates, including alkyl ether carboxylates (such as sodium laureth carboxylate); acyl lactylates (such as sodium cocoyl lactylate); alkanoyl sarcosinates (such as sodium lauroyl sarcosinate, sodium cocoyl sarcosinate, and ammonium lauroyl sarcosinate); alkylglyceryl ether sulfonates (such as sodium cocoglyceryl ether sulfonate); and olefin sulfonates (such as sodium C₁₄₋₁₆ olefin sulfonates). Suitable nonionic surfactants include, for example, compounds produced by the condensation of alkylene oxide with an organic hydrophobic compound which can be either aliphatic, alicyclic or aromatic in structure. Nonionic surfactants are illustrated by polyethylene oxide condensates of C₆-C₁₂ alkylphenols; condensates of ethylene oxide with the reaction product of propylene oxide and ethylenediamine; long chain tertiary amine oxides; long chain tertiary phosphine oxides; long chain dialkyl sulfoxides; and the like. Suitable cationic surfactants are compounds containing positively charged amine or quaternary ammonium groups. Suitable amphoteric surfactants include derivatives of aliphatic quaternary ammonium, phosphonium and sulfonium compounds. One class of amphoteric surfactants are zwitterionic compounds such as betaines, sultaines and phosphobetaines. Illustrative of a betaine is cocoamidopropyl betaine. Another class of amphoteric surfactants are compounds containing an amine group and an anionic group such as carboxylate, sulfonate, sulfate, phosphate or phosphonate, as illustrated by sodium 3-dodecylaminopropionate and sodium 3-dodecylaminopropane sulfonate.

The acne cleanser composition can also contain skin-benefitting agents (such as, for example, exfoliants, hydrators, skin brighteners, humectants, skin soothers, antifungals, antimicrobials, and the like). Suitable skin benefiting agents are known to those skilled in the art. In addition, the acne cleanser composition can contain ingredients to improve the feel and presentation of the composition to the user (such as, for example, viscosity boosters, pearlizing agents, thickeners, preservatives, fragrance, water softener, colorants, and the like). These ingredients are conventional and well known to those skilled in the art.

The second composition used in the regimen in accordance with this disclosure is an acne spot treatment composition that also contains an anti-acne active ingredient. In addition to the anti-acne active ingredient, the acne spot treatment composition can also contain skin-benefitting agents (such as, for example, exfoliators, pore reducing agents, hydrators, skin healers, humectants, moisturizers, circulation aids, oil absorbers, skin soothers, antifungals, antimicrobials, natural botanicals, and the like). Suitable skin benefiting agents are known to those skilled in the art. In addition, the acne spot treatment composition can contain ingredients to improve the feel and presentation of the composition to the user (such as, for example, solubilizers, neutralizers, thickeners, preservatives, fragrance, colorants, and the like). These ingredients are conventional and well known to those skilled in the art.

The third composition used in the regimen in accordance with this disclosure is a hydrating day cream composition. The hydrating day cream composition contains an anti-acne active ingredient and a combination of farnesol and sodium hyaluronate. The farnesol and sodium hyaluronate can be present in the hydrating day cream in a ratio in the range of 30 parts farnesol to 1 part sodium hyaluronate to 10 parts farnesol to 1 part sodium hyaluronate. In particularly useful embodiments, farnesol and sodium hyaluronate are present in a ratio of 15:1. The total amount of famesol and sodium hyaluronate together can be in the range of 0.1 weight percent to 100 weight percent based on the total weight of the hydrating day cream composition. In particularly useful embodiments, total amount of farnesol and sodium hyaluronate together can be in the range of 0.3 weight percent to 10 weight percent based on the total weight of the hydrating day cream composition. In addition to the anti-acne active ingredient, farnesol and sodium hyaluronate, the hydrating day cream can also contain skin-benefitting agents (such as, for example, exfoliators, skin brighteners, sebum control agents, pore reducing agents, hydrators, skin healers, humectants, moisturizers, circulation aids, oil absorbers, skin soothers, antifungals, antimicrobials, natural botanicals, and the like). Suitable skin benefiting agents are known to those skilled in the art. In addition, the hydrating day cream composition can contain ingredients to improve the feel and presentation of the composition to the user (such as, for example, solubilizers, neutralizers, thickeners, preservatives, water softener, fragrance, colorants, and the like). These ingredients are conventional and well known to those skilled in the art.

The fourth composition used in the regimen in accordance with this disclosure is an acne night cream composition. The acne night cream composition also contains an anti-acne active ingredient and a combination of famesol and sodium hyaluronate. The farnesol and sodium hyaluronate can be present in the night cream in a ratio in the range of 100 parts farnesol to 1 part sodium hyaluronate to 40 parts famesol to 1 part sodium hyaluronate. In particularly useful embodiments, farnesol and sodium hyaluronate are present in a ratio of 60:1. The total amount of farnesol and sodium hyaluronate together can be in the range of 0.1 weight percent to 100 weight percent based on the total weight of the acne night cream composition. In particularly useful embodiments, total amount of farnesol and sodium hyaluronate together can be in the range of 0.3 weight percent to 10 weight percent based on the total weight of the acne night cream composition. In addition to the anti-acne active ingredient, farnesol and sodium hyaluronate, the acne night cream can also contain skin-benefitting agents (such as, for example, exfoliators, skin brighteners, pore reducing agents, hydrators, skin healers, humectants, moisturizers, circulation aids, oil absorbers, skin soothers, antifungals, antimicrobials, natural botanicals, and the like). Suitable skin benefiting agents are known to those skilled in the art. In addition, the acne night cream composition can contain ingredients to improve the feel and presentation of the composition to the user (such as, for example, solubilizers, neutralizers, thickeners, preservatives, fragrance, colorants, and the like). These ingredients are conventional and well known to those skilled in the art.

The methods of treating acne in accordance with this disclosure include the steps of: a) washing the skin of a person having acne with the above-described acne cleanser; b) applying the above-described acne spot treatment to acne blemishes on the skin of the person afflicted with acne; c) applying the above-described hydrating day cream to the skin of the person afflicted with acne; and d) applying the above-described acne night cream to the skin of the person afflicted with acne. The acne treatment regimen in accordance with tis disclosure results in the reduction of inflammatory lesions, a decrease in inflammation, a noticeable reduction of oiliness, and an excellent reduction of new lesions.

The following examples are included for illustrative purposes only and are not intended to limit the scope of the invention.

### EXAMPLE

An acne cleanser composition is prepared in accordance with this disclosure. The following ingredients mixed in a kettle with heating to 73° C. to prepare “Phase A” of the acne cleanser composition:

Phase A

| Ingredient | Weight % |
|---|---|
| Water | 31.00 |
| Green Tea Extract | 0.01 |
| Algae Extract | 0.01 |
| Ginsing Extract | 0.01 |
| Ginko Biloba Extract | 0.01 |
| Panthenol | 0.01 |
| Papain | 0.00001 |
| Structure Plus (a thickener | 8.00 |
| commercially available from Natural | |
| Starch, Bridgewater, NJ) | |
| Sodium Citrate Granules | 1.80 |
| Lactic Acid | 0.10 |
| Ascorbic Acid | 0.01 |
| Ammonium Hydroxide | 0.03 |
| Tetrasodium EDTA | 0.10 |
| Sodium Lauroyl Sarcosinate | 10.00 |
| Cocamidopropyl Betaine | 5.00 |

In a kettle equipped with a Lightin type mixer, the ingredients of Phase A were added in order with heating to 73° C.

Phase B

| Ingredient | Weight % |
|---|---|
| Cocamidopropyl Betaine | 10.00 |
| Phospholipid EFA (available from Mona Indus., | 2.00 |
| Patterson, NJ) | |
| Standamox CAW (available from Henkel Corp., | 5.00 |
| Gulf Mills, PA) | |
| Salicylic Acid | 2.00 |
| Vitamin E Acetate | 0.00001 |
| Bio-Terge AS-40 (available from Stepan Co., Northfield, IL) | 20.00 |

Phase C

| Ingredient | Weight % |
|---|---|
| Glycol Stearate | 2.00 |

In a separate kettle, Phase B ingredients were added together in order and the mixture heated to 60° C. with mixing, but avoiding aeration. Phase B was added to Phase A at 73° C. with mixing. At 73° C. Phase C is added, mixed well then cooled to 45° C.

Phase D

| Ingredient | Weight % |
|---|---|
| Apple Extract (available from Active Organics, | 0.01 |
| Lewisville, TX) | |
| Cola Extract (available from Active Organics Lewisville, TX) | 0.01 |
| Propylene Glycol | 0.01 |
| Bisabolol (available from Dragoco, Totowa, NJ) | 0.01 |
| Phospholipids (available from Mona Indus., Patterson, NJ) | 0.01 |
| Fragrance (Ungerer Blend from Ungerer & Co., | 0.50 |
| Lincoln Park, NJ) | |

Phase D was added and mixed until uniform. Phase E was premixed and added to the batch with mixing. Then the batch was cooled.

Phase E

| Ingredient | Weight % |
|---|---|
| Water | 2.00 |
| Germall II (available from ISP, Lombard, IL) | 0.30 |
| Kathon CG | 0.06 |
| (available from Rohm and Haas Co., North Olmstead, OH) | |

Phase F

| Ingredient | Weight % |
|---|---|
| Blue #1 | QS |

At 45° C. the Phase D ingredients were added. Then in a separate kettle, Phase E was pre-mixed and added at 45° C. Phase F was then added and the batch cooled.

An acne spot treatment composition in accordance with the present disclosure was prepared. The indicated ingredients were combined with mixing and heating to 45° C. to prepare “Phase A”.

Ingredients

| Phase | Percent | Ingredient |
|---|---|---|
| A | 51.45 | Water |
| A | 0.10 | Echinacea extract |
| A | 0.10 | Willowherb extract |
| A | 0.10 | Green Tea extract |
| A | 0.10 | Yucca extract |
| A | 0.10 | Lavender Extract |
| A | 0.10 | Ormagel SH |
| A | | (available from Assessa Industries, |
| | | Rio de Janeiro, Brazil) |
| A | 0.01 | Licorice Extract |
| A | 0.10 | Panthenol |
| A | 0.01 | Bliodyne TRF 25% SOLUTION |
| A | | (available from Brooks Industries, South Plainfield, NJ) |
| A | 5.00 | Witch Hazel Distillate |
| A | 4.00 | Butylene Glycol |
| A | 0.80 | Rhodigel |
| A | | (available from Rhodia Inc., Cranbury, NJ) |
| A | 0.10 | Versene 100XL (available from Dow Chemical USA, |
| | | Midland, MI) |
| A | 0.02 | Ascorbic Acid |
| A | 0.05 | Biomin Aquacinque #06342 |
| | | (available from Brooks Industries, South Plainfield, NJ) |
| A | 0.50 | Vegepol |
| | | (available from Brooks Industries, South Plainfield, NJ) |
| A | 0.10 | Sebustop (available from Solabia, Cedex, France) |
| A | 0.10 | Niacinamide |
| A | 1.5 | Hysol BT (1%) (available from Centre Chem, |
| | | Stamford, CT) |
| A | 3.41 | Lactic Acid (88%) |
| A | 1.2 | Ammonium Hydroxide |
| A | 0.1 | Prodow 400 (available from Ajinomoto, Paramus, NJ) |
| B | 25.00 | SD Alcohol 40 |
| B | 2.00 | Salicylic Acid |
| C | 2.25 | Solubilisant (available from L.C.W., Sao Paulo, Brazil) |
| C | 0.30 | Farnesol |
| C | 0.25 | Natural Chamomile |
| C | 0.15 | Aromaphyte of Lemongrass |
| C | 0.0001 | Apple Extract |
| C | 0.0001 | Cola Extract |
| C | 0.0001 | Propylene Glycol |
| C | 0.0001 | Bisabolol (available from Dragoco, Totowa, NJ) |
| C | 0.0001 | Phospholipids (available from Mona Indus., |
| | | Patterson, NJ) |
| C | 0.0001 | Vitamin E Acetate |
| C | 0.0001 | Vitamin A Palmitate |
| D | 1.0 | Silica |
| E | QS | Blue 1 |
| E | QS | Violet 2 |

The SD Alcohol and Salicylic Acid were pre-mixed and added to Phase A. Next, Phase C was prepared by mixing the Phase B ingredients listed above. After Phase C was added to the main mixture, fumed Silica was added and the mixture homogenized. Finally, Blue 1 and Violet 2 were added.

A hydrating day cream composition in accordance with this disclosure is prepared. For this formulation, all percentages are weight percent based on the total weight of the total composition. The following ingredients are mixed with heating to 75° C. to make Phase A:

Phase A

| Ingredient | Weight % |
|---|---|
| Arlacel 165 (available from I.C.I., Wilmington, MA) | 1.5 |
| Promulgen “G” (available from Croda Inc., Parsippany, NJ) | 1.0 |
| Arlacel 60 (available from I.C.I., Wilmington, MA) | 0.5 |
| Montanov 202 (available from Seppic, Fairfield, NJ) | 2.5 |
| Silicone 556 (available from Dow Chemical | 1.5 |
| USA. Midland, MI) | |
| Probutyl 14 (available from Croda Inc., Parsippany, NJ)) | 3.25 |
| Finsolve EMG-20 (available from Finetex, | 1.0 |
| Elmwood Park, NJ) | |
| Salicylic Acid | 1.0 |
| Propylparaben | .03 |

Phase B was prepared by adding 0.4% Veegum K (available from Vanderbilt, Norwalk, Conn.) to water (60.33%). Once the Veegum was dispersed, 0.2% Panthenol and 0.09% Allantoin were added with continued mixing. Phase C (a pre-mix of 5% Pentylene Glycol, 0.45% Rhidogel and 0.15% PCG-10 (available from Aqualon, Wilmington, Del.)) was then added to Phase B with mixing and heating to 75° C. Phase A was then mixed with Phase B/C mixture for 2 to 3 minutes. This main mixture was then deaerated and cooled to 60° C. Then the following Phase D ingredients were added.

Phase D

| Ingredient | Weight % |
|---|---|
| Aloe Powder | .001 |
| Green Tea Extract | 0.1 |
| Grapeseed Extract | 0.1 |
| Prodew 400 (available from Ajinomoto, Paramus, NJ) | 0.5 |
| Ormagel SH (available from Assessa Industries, | 0.5 |
| Rio de Janeiro, Brazil) | |
| Urea | 3.0 |
| Hampene 100 XL (available from Dow | 0.1 |
| Chemical USA. Midland, MI) | |
| Ascorbylsilane “C” (available from Eskymol) | 0.1 |

Phase E (a pre-mix of butylene glycol (3.0%) and methylparaben (0.05%)) were then added to the main mixture. Next, Phase F is added to the main mixture with continued stirring.

Phase F

| Ingredient | Weight % |
|---|---|
| Hysol BT (1%) (available from Centre Chem, Stamford, CT) | 2.0 |
| Sebustop (available from Solabia, Cedex, France) | 0.10 |
| Biopol HE (available from Brooks Industries, | 0.1 |
| South Plainfield, NJ) | |
| Apple Extract (available from Active Organics | 0.0001 |
| Lewisville, TX) | |
| Cola Extract (available from Active Organics Lewisville, TX) | 0.0001 |
| Propylene Glycol (available from Dow | 0.0001 |
| Chemical USA. Midland, MI) | |
| Bisabolol (available from Dragoco, Totowa, NJ) | 0.0001 |
| Phospholipids (available from Rona Corp., Hawthorne, NY) | 0.0001 |
| Biomin TRF 25 (available from Brooks Indus., | 0.1 |
| South Plainfield, NJ) | |
| Ungerer Chamomile Blend | 0.4 |
| Lemongrass Actiphyte | 0.2 |
| Farnesol (available from Dragoco, Totowa, NJ) | 0.3 |

Phase G

| Ingredient | Weight % |
|---|---|
| Dry Flo Powder AF (available from | 5.00 |
| National Starch, Chicago, IL) | |

The batch was cooled to 45° C. and Phase G added with mixing.

Phase H

| Ingredient | Weight % |
|---|---|
| Water | 3.00 |
| Germall II (available from ISP, Lombard, IL) | 0.20 |

Phase H was pre-mixed until soluble and added to the batch with mixing. Coloring was added and the batch cooled to room temperature.

An acne night cream composition in accordance with this disclosure was prepared. For this formulation, all percentages are weight percent based on the total weight of the total composition. The following ingredients were mixed with heating to 75° C. to make Phase A.

Phase A

| Ingredient | Weight % |
|---|---|
| Arlacel 165 (available from I.C.I., Wilmington, MA)) | 2.0 |
| Promulgen G (available from Croda Inc., Parsippany, NJ) | 1.0 |
| Arlacel 60 (available from I.C.I., Wilmington, MA) | 0.5 |
| Montanov 202 (available from Seppic, Fairfield, NJ) | 2.5 |
| Silicone 556 (available from Dow Chemical USA. | 1.0 |
| Midland, MI) | |
| ProButyl 14 (available from Croda Inc., Parsippany, NJ) | 5.0 |
| Finsolv EMG-20 (available from Finetex, Elmwood Park, NJ) | 5.0 |
| Vitamin E Acetate | 0.1 |
| Vitamin A Palmitate | 0.1 |
| Salicylic Acid | 2.0 |
| Propylparaben | .03 |
| Bio Oil HBSL (available from Brooks Indus., | 0.5 |
| South Plainfield, NJ) | |

Phase B was prepared by adding the following ingredients to a pre-mix containing 56.66% water and 0.75% VEEGUM K (commercially available from Vanderbilt, Norwalk, Conn.).

Phase B (Continued)

| Ingredient | Weight % |
|---|---|
| Green Tea Extract | 0.10 |
| Allantoin | .09 |
| Panthenol | 0.3 |
| Aloe Powder | .01 |
| Prodew 400 (available from Ajinomoto, Paramus, NJ) | 1.0 |
| Niacinamide | 0.1 |
| Ammonium Hydroxide | 1.5 |
| Hysol BT (1%) (available from Centre Chem, Stamford, CT) | 0.5 |
| Hampene 100XL (available from Dow Chemical | 0.1 |
| USA. Midland, MI) | |
| Lactic Acid | 3.41 |
| Ascorbylmethylsilanol Pectinate | 0.1 |

Then, a pre-mix containing 5.0% Pentylene Glycol, 0.05% Methylparaben, 0.45% Rhidogel (commercially available from Vanderbilt, Norwalk, Conn.), and 0.15% Cellosize PCG-10 (commercially available from (available from Aqualon, Wilmington, Del.) was added with mixing and heating to 75° C.

Phase A was added to Phase B and mixed for 2-3 minutes. The mixture was de-aerated and cooled to 45° C. The ingredients of Phase C (see list below) were then added with mixing. Then, 5% Dry Flo AF (available from Natural Starch, Bridgewater, N.J.) was added. A mixture of 3% water and 0.2% GERMALL II (available from ISP, Lombard, Ill.) was then added. Finally dye (Blue #1) was added to provide a desired color.

Phase C

| | Weight |
|---|---|
| Ingredient | % |
| Biomin TRF-25 (available from Brooks Indus., | .25 |
| South Plainfield, NJ) | |
| Biopol OE (available from Brooks Indus., South Plainfield, NJ) | 0.2 |
| Sebustop (available from Solabia, Cedex, France) | 0.2 |
| Apple Extract (available from Active Organics, Lewisville, TX) | 0.02 |
| Cola Extract (available from Active Organics, Lewisville, TX) | 0.02 |
| Propylene Glycol (available from Dow Chemical | 0.02 |
| USA. Midland, MI) | |
| Bisabolol (available from Dragoco, Totowa, NJ) | 0.02 |
| Phospholipids (available from Mona Corporation) | 0.02 |
| Fragrance (Ungerer Blend available from Ungerer & Co.) | 0.5 |
| Aromaphyte of Lemongrass (Active Organics, Lewisville, TX) | 0.25 |
| Farnesol (available from Dragoco, Totowa, NJ) | 0.3 |

The foregoing compositions were used in an acne treatment regimen in accordance with the present disclosure. Specifically, a study was undertaken to determine the efficacy of the foregoing facial products for the treatment of active acne. After a five day washout period, twenty participants (who were between the ages of 15 and 40) were instructed not to use any other skin care products during the course of the study. The patients were afflicted with: mild to moderate acne or rosacea; acne resulting in closed and open comedones, erythematous pustules, diffuse erythema; and/or Inflammatory redness. Participants were excluded if during the study they used: any acne topical and/or oral medication; any other skin care products during the study; any cosmetic procedures such as peels, collagen, etc. at least one month before the study; or if they had any chronic skin conditions such as eczema, psoriasis, severe sun damage. The participants were instructed to use the products as follows:

i. In the morning, use the acne wash as directed on the container and then apply the acne treatment on the entire face as directed follow by the hydrator and sunscreen. The spot treatment may be used on any active lesions.

ii. In the evening, use the acne wash followed by the night cream which should be applied on the entire face. The spot treatment may be used on any active lesion.

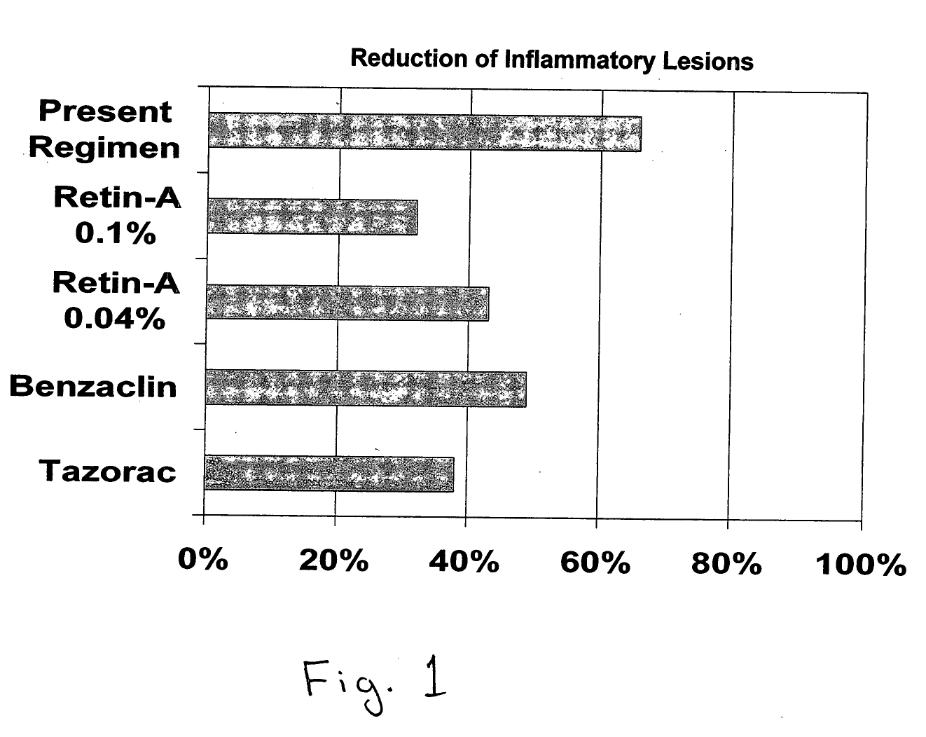

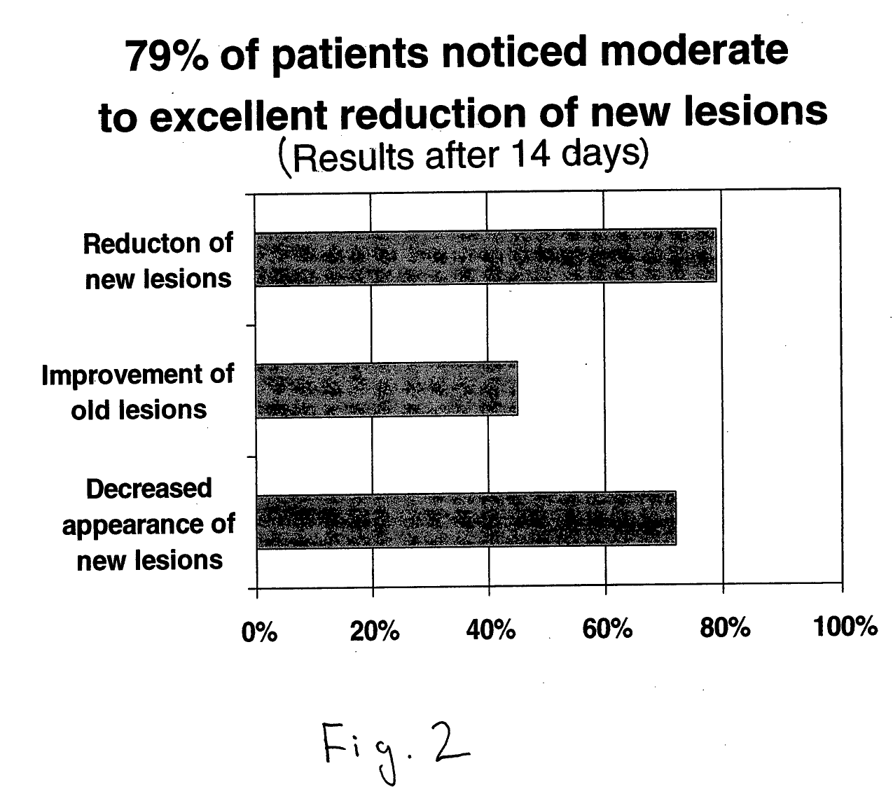

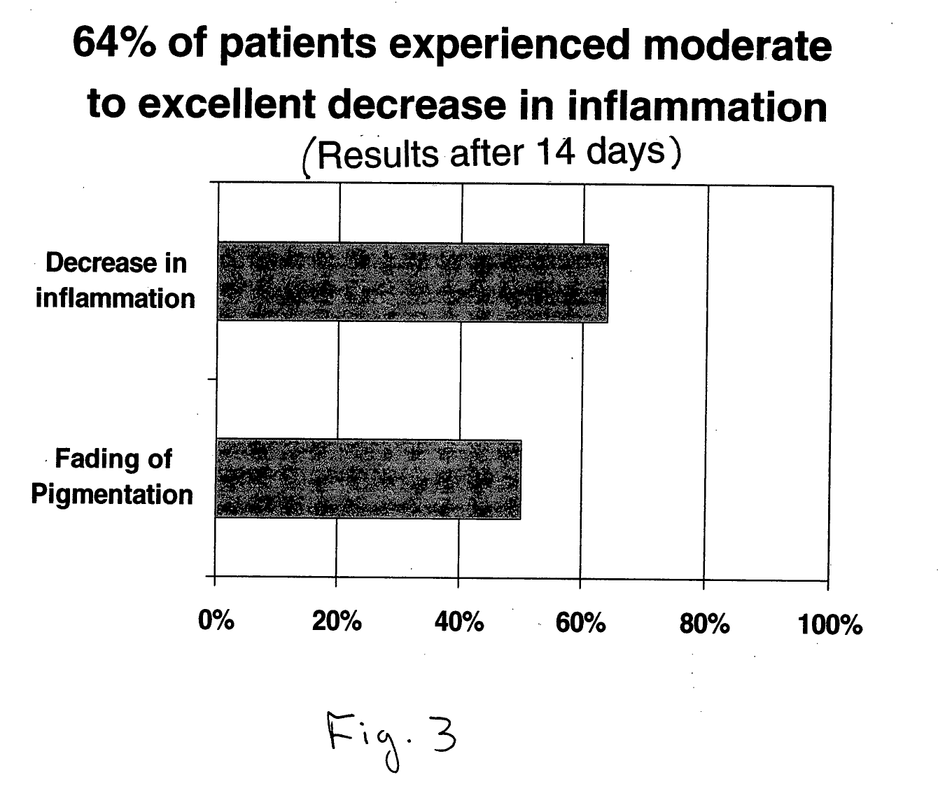

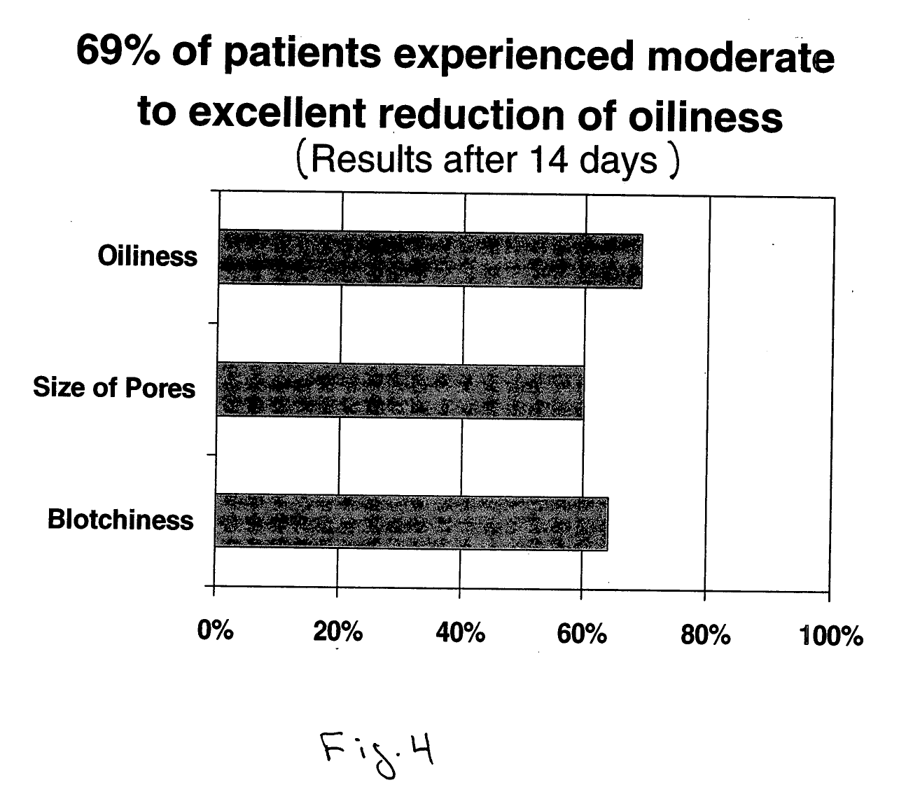

The individuals were observed periodically over the course of the treatment and the degree of oiliness, inflammation, amount of inflammatory lesions, and the amount of new lesions were observed. For comparison, treatment with 0.1% Retin-A, 0.04% Retin-A, Benzaclin (commercially available from Ortho Labs), and Tazorac (commercially available from Ortho Labs) were also evaluated. As seen in FIG. 1 through 4, the present regimen provided excellent results compared to the other treatments.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended herein.

We claim:

1. An acne treatment regimen comprising the steps of:
a) cleansing the skin of an individual afflicted with acne with an acne cleanser composition containing an anti-acne active;
b) applying an acne spot treating composition to at least one lesion on the skin of the individual, the acne spot treating composition containing an anti-acne active ingredient;
c) applying a hydrating day cream composition to the skin of the individual, the hydrating day cream composition containing an anti-acne active ingredient and a combination of famesol and sodium hyaluronate; and
d) applying an acne night cream composition to the skin of the individual, the acne night cream composition containing an anti-acne active ingredient and a combination of farnesol and sodium hyaluronate.

2. A kit for the treatment of acne comprising:
a) a container of an acne cleanser composition containing an anti-acne active;
b) a container of an acne spot treating composition containing an anti-acne active ingredient;
c) a container of a hydrating day cream composition containing an anti-acne active ingredient and a combination of farnesol and sodium hyaluronate; and
d) a container of an acne night cream composition containing an anti-acne active ingredient and a combination of farnesol and sodium hyaluronate.

3. A composition comprising:
an anti-acne active ingredient and a combination of farnesol and sodium hyaluronate.
